(19) United States
(12) Patent Application Publication
(10) Pub. No.: US 2025/0277275 A1
(43) Pub. Date: Sep. 4, 2025

(54) SET OF PRIMERS, COMPOSITION OF REAGENTS AND METHOD OF DETECTING ATYPICAL BACTERIA

(71) Applicant: GENOMTEC S.A., Wroclaw (PL)

(72) Inventors: Miron TOKARSKI, Brzeg (PL); Izabela PIELKA, Konopiska (PL); Malgorzata MALODOBRA-MAZUR, Wroclaw (PL)

(21) Appl. No.: 18/255,991

(22) PCT Filed: Dec. 8, 2021
(86) PCT No.: PCT/PL2021/050085

§ 371 Date: Jun. 5, 2023

(30) Foreign Application Priority Data
Dec. 8, 2020 (PL) P.436263

(51) Int. Cl. C12Q 1/689; C12Q 1/6844

(52) U.S. Cl. CPC C12Q 1/689; C12Q 1/6844

(57) ABSTRACT

The invention relates to a set of primers for amplifying the nucleotide sequence of the Chlamydophila pneumoniae bacterium MOMP gene, a method for detecting Chlamydophila pneumoniae bacteria, a method for detecting an infection caused by Chlamydophila pneumoniae bacteria, and a kit for detecting an infection caused by Chlamydophila pneumoniae bacteria.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Nov. 29, 2023, is named PZ8026USAW s125 ST25.txt and is 2,590 bytes in size.

The present invention relates to a set of primers for amplifying the nucleotide sequence of the Chlamydophila pneumoniae MOMP gene, a method for detecting Chlamydophila pneumoniae bacteria, a method for detecting an infection caused by the Chlamydophila pneumoniae bacteria, and a kit for detecting a Chlamydophila pneumoniae infection. The invention is applicable in medical diagnostics.

Chlamydophila pneumoniae belongs to the obligate intracellular bacteria, distinguished by a specific two-phase development cycle. It consists of two successive forms: elementary and reticulate bodies. The former are a metabolically inactive form that infects the host. In the process of endocytosis, they get inside the cell, where they transform into actively metabolic, non-infectious reticulate bodies. After 48-72 hours, condensation, reorganization, and division of reticulate bodies into elementary ones take place. Elementary bodies accumulate in cells in the amount of 500 to 1000. Despite their large number, they do not affect the functioning of the host cells. A cell filled with bodies induces the release of chlamydia antigens outside the cell, thereby triggering an immune response. Due to the ongoing cycle inside the cells, it is impossible to cultivate them on microbiological media. Diagnosis of Chlamydophila pneumoniae infections is thereby difficult. Chlamydophila pneumoniae can cause both lower and upper respiratory tract infections. Mainly causing pneumonia and bronchitis. In the case of upper respiratory tract, the infections involve inflammation of the sinuses or pharynx, either isolated or in conjunction with a lower respiratory tract infection. Incubation of infection takes approximately 21 days.

Currently used diagnostic methods are insufficiently sensitive and specific. Commonly used methods include serological and immunological (EIA, methods. ELISA) IgM antibodies are detectable about 3 weeks after infection, and IgG antibodies may arise 6-8 weeks after symptoms appear. Both immunological and serological methods are not sufficient for a rapid and precise diagnosis of Chlamydophila pneumoniae. Microbiological methods, like inoculating culture media is impossible without the use of cells since the pathogen is intracellular. The methods characterized by the greatest specificity and sensitivity are those involving the detection of Chlamydophila pneumoniae nucleic acid in biological material (the so-called NAAT methods—Nucleic Acid Amplification Tests), i.e., in throat swabs or nasopharyngeal swabs. The most commonly used tests in NAAT technology are Real-Time PCR-based assays. Many different tests using the Real-Time PCR technique are available on the market, but despite the fierce competition, these methods are still relatively expensive. Moreover, they require highly specialized personnel, expensive devices, and extraction of genetic material from the patient's sample is necessary. Moreover, since cyclic heating and cooling of the reagents is necessary, this method is relatively long, and the devices used use relatively much energy to carry out this process.

Isothermal methods, including LAMP (Loop-mediated isothermal amplification) method, are methods that allow to speed up the diagnostic process, reduce the cost of energy needed to perform the analysis and allow the determination to be performed with no need to extract DNA/RNA of the detected pathogen. Moreover, according to the literature data, these methods are characterized by higher sensitivity and specificity than the aforementioned Real-Time PCR technique, they are also much faster. The isothermal course does not require specialized equipment. Due to the low hardware requirements, isothermal methods are an ideal diagnostic solution for primary care units (POCT-point-of-care testing), where the test can be performed in the practice of a general practitioner or medical specialist (ENT, pediatrician) at the first contact of a patient with the doctor. This solution allows for a quick turnaround (in no more than 15 minutes), which allows for introduction of a targeted therapy during the very first visit. This is especially important with atypical bacteria such as Chlamydiophila pneumoniae. On the other hand, the use of freeze-dried reagents allows the tests to be stored at room temperature, without the need to freeze the diagnostic tests.

The use of primers in the LAMP method for the diagnosis of Chlamydophila pneumoniae is known from the patent applications published so far: CN111378769A; CN101886122A; CN104818333A; CN107099619A; KR20160088316A. The LAMP method is disclosed, for example, in patent specifications WO0028082, WO0224902. The above-mentioned patent applications in most cases do not describe the sensitivity and detection limit of Chlamydophila pneumoniae bacteria or these limits are higher than in the present invention. Some of them do not contain information on the assay specificity. The detection method in some of the above-mentioned patent applications uses larger volumes of reaction mixtures, does not allow for quantitative measurement, and the detection is of the end-point type, using agarose gel electrophoresis or other methods based on changing the color of the reaction mixture upon a positive result of the amplification reaction (SYBR Green, Hydroxy-Naphthol-Blue, cresol red). Moreover, most of the kits developed and described above are not applicable in POCT diagnostics, and their main application is in laboratories. Therefore, there is still a need to provide a diagnostic method using appropriately refined sets of primers used for the diagnosis of Chlamydiophila pneumoniae with the LAMP method, intended for use in point-of-care testing, which allows the detection of bacteria with an extremely low detection limit (≥5 copies/reaction) in a short time (≤15 min). Unexpectedly, the above problem was solved by the present invention.

The first subject of the invention is a set of primers for amplifying the nucleotide sequence of the Chlamydophila pneumoniae MOMP gene, characterized in that it contains a set of internal primers with the following nucleotide sequences a) and b), as well as a set of external primers containing the following nucleotide sequences c) and d) specific for the selected fragment of the Chlamydophila pneumoniae MOMP gene:
a) 5′ CGCTCCAAGAGAAAGAGGTGTC 3′ (nucleic sequence SEQ ID NO: 3 or its reverse and complementary sequence, linked from the 3′ end, preferably by a TTTT bridge, to the sequence 5′ AAACGTTTCTTTAAGTAACGGAG 3′-(nucleic sequence SEQ ID NO: 4 or its reverse and complementary sequence;
b) 5′ CTCGTGGAGCCTTATGGGAA 3′-(nucleic sequence SEQ ID NO: 5 or its reverse and complementary sequence, linked from the 3′ end, preferably by bridge, to a TTTT the sequence 5′ GTGCATATTGGAATTCAGCT 3′-(nucleic sequence SEQ ID NO: 6 or its reverse and complementary sequence;
c) 5′ CTGTAAATGCAAATGAACTACC 3′ nucleic sequence SEQ ID NO: 1 or its reverse and complementary sequence, and
d) 5′ CACATTAAGTTCTTCAACTTTAGGT 3′ nucleic sequence SEQ ID NO: 2 or its reverse and complementary sequence.

In a preferred embodiment of the invention, the primer set comprises a loop primer sequence comprising nucleic sequences contained in or complementary to the Chlamydophila pneumoniae MOMP gene SEQ ID NO: 7-5′ TGCGGTTGTGCAACTTTGGG 3′ or a sequence reverse and complementary thereof.

The second subject of the invention is a method for detecting Chlamydophila pneumoniae bacteria, characterized in that a selected region of the nucleotide sequence of the Chlamydophila pneumoniae genome (MOMP gene fragment) is amplified using a primer set as defined in the first subject of the invention, the amplification method being the LAMP method.

In a preferred embodiment, the amplification is carried out with a temperature profile of: 65.5° C., 40 min. By temperature profile is meant that the amplification is carried out at a constant temperature of 65.5° C. for a period of 40 minutes.

In a further preferred embodiment of the invention, the end-point reaction is carried out with a temperature profile of 80° C., for additional 5 min.

The third subject of the invention is a method for detecting an infection caused by the Chlamydophila pneumoniae bacterium, characterized in that it comprises the detection method defined in the second subject of the invention.

The fourth subject of the invention is a kit for the detection of an infection caused by Chlamydophila pneumoniae, characterized in that it comprises a set of primers as defined in the first subject of the invention.

In a preferred embodiment of the invention, the infection detection kit comprises 5.0 μl of WarmStart LAMP 2X Master Mix (New England Biolabs website).

In a further preferred embodiment of the invention, individual amplification primers as defined in the first subject of the invention, the primers having the following concentrations: 0.13 μM F3, 0.13 μM B3, 1.06 μM FIP, 1.06 μM BIP, 0.27 μM LoopB; D-(+)-Trehalose dihydrate—6%; fluorescent marker interacting with double-stranded DNA-EvaGreen (Biotium website) in the amount of ≤0.5 μl or GreenFluorescent Dye (Lucigen website) in the amount of ≤1 μl or Syto-13 (ThermoFisher Scientific website) ≤16 μM or SYTO-82 (ThermoFisher Scientific website) ≤16 μM or another fluorescent dye interacting with double-stranded DNA at a concentration that does not inhibit the amplification reaction.

The advantage of the primer sets of the invention for the detection of Chlamydophila pneumoniae, as well as the method for detecting Chlamydophila pneumoniae infection and the method of detecting the amplification products is the possibility of using them in medical diagnostics at the point of care (POCT) in the target application with a portable genetic analyzer. Freeze-drying of the reaction mixtures of the invention allows the diagnostic kits to be stored at room temperature without reducing the diagnostic parameters of the tests. In turn, the use of a fluorescent dye to detect the amplification product increases the sensitivity of the method, allows to lower the detection limit (down to 1 copy/reaction), as well as it enables the quantitative measurement of bacteria in the test sample.

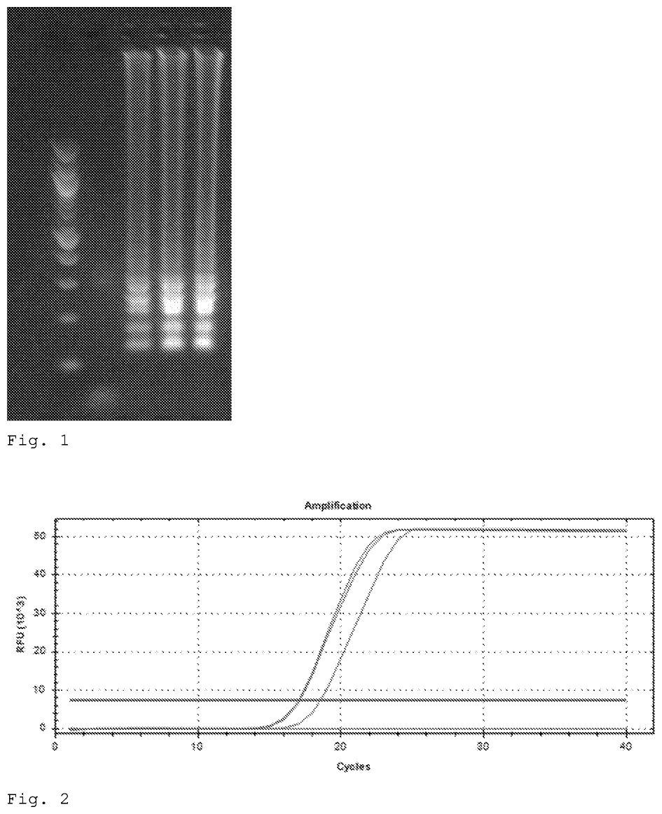

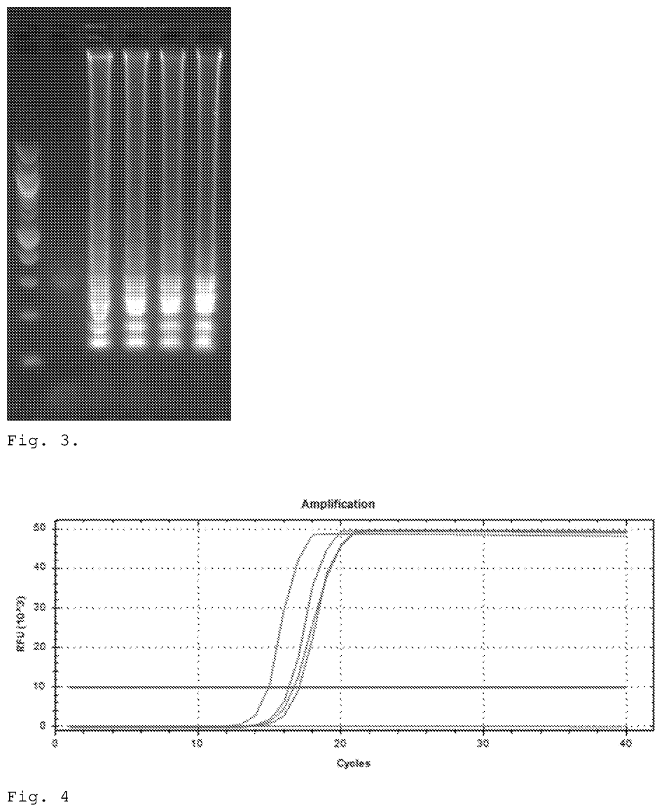

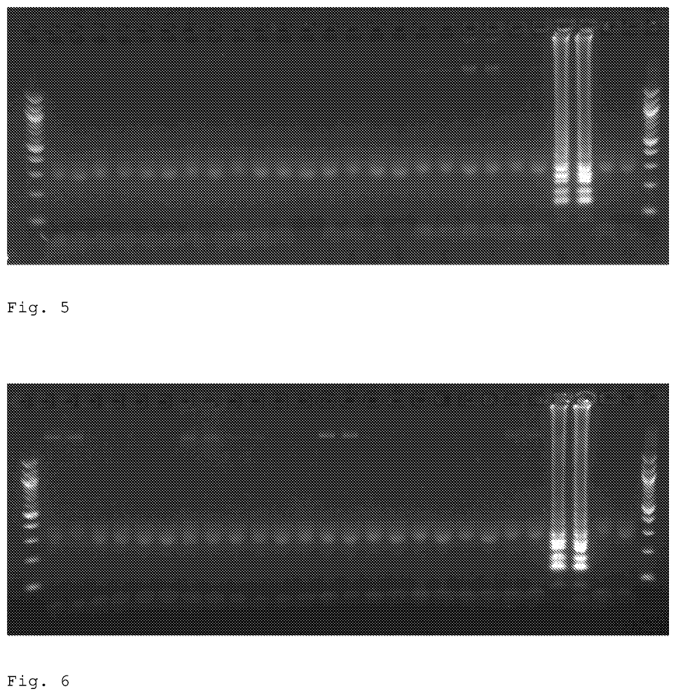

Exemplary embodiments of the invention are presented in the drawing, in which FIG. 1 shows the sensitivity characteristics of the method, where a specific signal was obtained with the template: Genomic DNA from Chlamydophila pneumoniae Strain: CM-1 (VR-1360DQ™) over the range of 10-1 copies of the DNA standard/reaction, but there was no product in NTC; FIG. 1: lane 1: mass marker (Quick-Load® Purple 100 bp DNA Ladder, NewEngland Biolabs); lane 2: NTC; lane 3:1 copy of ChP; lane 4:5 copies of ChP; lane 5:10 copies of ChP; FIG. 2 shows the sensitivity of the method of the invention measured by assaying a serial dilution of the Genomic DNA from Chlamydophila pneumoniae Strain: CM-1 (VR-1360DQ™) standard over a range of 10-1 DNA standard copies/reaction, where the product amplification was measured in real time. The results of the real-time Chlamydophila pneumoniae detection are shown in Table 1, giving the minimum time required to detect the fluorescence signal; FIG. 3 shows the sensitivity characteristics of the method, where a specific signal was obtained with the template: Genomic DNA from Chlamydophila pneumoniae Strain: CM-1 (VR-1360DQ™) over the range of 100-10 copies of the DNA standard/reaction, but there was no product in NTC; FIG. 3: lane 1: mass marker (Quick-Load® Purple 100 bp DNA Ladder, NewEngland Biolabs); lane 2: NTC; lane 3:10 copies of ChP; lane 4:25 copies of ChP; lane 5:50 copies of ChP; lane 6:100 copies of ChP; FIG. 4 shows the sensitivity of the method of the invention measured by assaying a serial dilution of the Genomic DNA from Chlamydophila pneumoniae Strain: CM-1 (VR-1360DQ™) standard over a range of 100-10 copies of the DNA standard/reaction, where the product amplification was measured in real time. The results the real-time Chlamydophila pneumoniae detection are shown in Table 2, giving the minimum time required to detect the fluorescence signal; FIGS. 5 and 6 show the specificity of the method of the invention with standard matrices of a number of pathogens potentially present in the tested biological material as natural physiological flora, those which may result from co-infections or those which share similar genomic sequences. FIG. 5: lane 1: mass marker (Quick-Load® Purple 100 bp DNA Ladder, NewEngland Biolabs); lanes 2 and 3: methicillin-sensitive Staphylococcus aureus (MSSA); lanes 4 and 5: Mycoplasma genitalium; lanes 6 and 7: Klebsiella pneumoniae; lanes 8 and 9: Bordetella pertussis; lanes 10 and 11: methicillin-resistant Staphylococcus aureus (MRSA); lanes 12 and 13: Streptococcus pyogenes; lanes 14 and 15: Enterococcus faecalis; lanes 16 and 17: Enterococcus faecium; lanes 18 and 19: Pseudomonas aeruginosa; lanes 20 and 21: Moraxella catarrhalis; lanes 22 and 23: Acinetobacter baumannii; lanes 24 and 25: Chlamydiophila pneumoniae; lanes 26 and 27: NTC; lane 28: mass marker (Quick-Load® Purple 100 bp DNA Ladder, NewEngland Biolabs), while FIG. 6: lane 1: mass marker (Quick-Load® Purple 100 bp DNA Ladder, NewEngland Biolabs); lanes 2 and 3: Listeria monocytogenes; lanes 4 and 5: Legionella pneumophila; lanes 6 and 7: Mycoplasma hominis; lanes 8 and 9: Haemophilus ducreyi; lanes 10 and 11: Escherichia coli; lanes 12 and 13: Ureoplasma urealyticum; lanes 14 and 15: Campylobacter jejuni; lanes 16 and 17: Candida albicans; lanes 18 and 19: Mycoplasma pneumoniae; lanes 20 and 21: Haemophilus influenzae; lanes 22 and 23: Human DNA; lanes 24 and 25: Chlamydiophila pneumoniae; lanes 26 and 27: NTC; lane 28: mass marker (Quick-Load® Purple 100 bp DNA Ladder, NewEngland Biolabs).

### EXAMPLE 1 PRIMER SEQUENCES

The sequences of specific oligonucleotides used for the detection of the Chlamydophila pneumoniae genetic material using LAMP technology are presented and characterized below.

1. The ChP MOMPF3 oligonucleotide sequence: 5′ CTGTAAATGCAAATGAACTACC 3′ (SEQ ID NO:1) is identical to the Chlamydophila pneumoniae MOMP gene (5′-3′ strand) which is 3′ end adjacent to the F2 primer.

2. The ChP MOMPB3 oligonucleotide sequence: 5′ CACATTAAGTTCTTCAACTTTAGGT 3′ (SEQ ID NO: 2) is a complementary fragment of the Chlamydophila pneumoniae MOMP gene (5′-3′ strand) 135 nucleotides away from the 3′ end of the oligonucleotide 1.

3. The ChP MOMPF2 oligonucleotide sequence: 5′ AAACGTTTCTTTAAGTAACGGAG 3′ (SEQ ID NO: 4) is identical to the Chlamydophila pneumoniae MOMP gene (5′-3′ strand) immediately adjacent to the 3′ end of the oligonucleotide 1.

4. The ChP MOMPB2 oligonucleotide sequence: 5′ GTGCATATTGGAATTCAGCT 3′ (SEQ ID NO: 6) is a complementary fragment of the Chlamydophila pneumoniae MOMP gene (5′-3′ strand) 108 nucleotides away from the 3′ end of the oligonucleotide 1.

5. The ChP MOMPF1c oligonucleotide sequence: 5′ CGCTCCAAGAGAAAGAGGTGTC 3′ (SEQ ID NO: 3) is a complementary fragment of the Chlamydophila pneumoniae MOMP gene (5′-3′ strand) 40 nucleotides away from the 3′ end of the 1 oligonucleotide.

6. The ChP MOMPB1c oligonucleotide sequence: 5′ CTCGTGGAGCCTTATGGGAA 3′ (SEQ ID NO:5) is identical to the Chlamydophila pneumoniae MOMP gene (5′-3′ strand) 68 nucleotides away from the 3′ end of the oligonucleotide 1.

7. The ChP MOMPLoopB oligonucleotide sequence: 5′ TGCGGTTGTGCAACTTTGGG 3′ (SEQ ID NO: 7).

The sequences of the F1c and F2 oligonucleotides are directly linked to each other or are preferably linked to each other by a TTTT bridge and used as FIP. The sequences of the B1c and B2 oligonucleotides are directly linked to each other or are preferably linked to each other by a TTTT bridge and used as BIP.

### Example 2. Composition of the Reaction Mixture

Method of amplifying the Chlamydophila pneumoniae MOMP gene using the oligonucleotides characterized in Example 1 with LAMP technology and the following composition of the reaction mixture:
5.0 μl WarmStart LAMP 2X Master Mix
0.13 μM F3
0.13 μM B3
1.06 μM FIP
1.06 μM BIP
0.27 μM LoopB
D-(+)-Trehalose dihydrate—6%
Fluorescent marker interacting with double-stranded DNA-EvaGreen ≤1X or Fluorescent dye 50X (New England Biolabs) in the amount of 0.5 μl or GreenFluorescent Dye (Lucigen) in the amount of ≤1 μl or Syto-13 ≤16 μM or SYTO-82 ≤16 μM or another fluorescent dye that interacts with double-stranded DNA at a concentration that does not inhibit the amplification reaction.
DNA template ≥1 copy/reaction

Total reaction volume adjusted to 10 μl with DNase and RNase free water.

### Example 3. Temperature Profile

Method of amplifying the Chlamydophila pneumoniae MOMP gene using the oligonucleotides characterized in Example 1 with LAMP technology and the composition of the reaction mixture characterized in Example 2 with the following temperature profile:
1) 65.5° C., 40 min
2) preferably for end-point reactions 80° C., 5 min.

### Example 4. Use of Fluorescent Dyes

Method of amplification and detection of the Chlamydophila pneumoniae MOMP gene using the oligonucleotides characterized in Example 1 with LAMP technology and the composition of the reaction mixture characterized in Example 2 with the temperature profile characterized in Example 3 and the detection method described below.

A fluorescent dye is used, capable of interacting with double-stranded DNA, added to the reaction mixture in an amount of 0.5 μl EvaGreen 20X; 0.5 μL or a concentration of ≤1X; ≤16 μM respectively for GreenFluorescent Dye (Lucigen); SYTO-13 and SYTO-82 before starting the reaction, real-time and/or end-point measurement. Excitation wavelength in the range similar to the FAM dye-490-500 nm (optimally 494 nm) for EvaGreen; Fluorescent dye 50X (New England Biolabs), GreenFluorescent Dye (Lucigen); SYTO-13 dyes and 535 nm (optimally 541 nm) for SYTO-82 dye; emission wavelength in the range 509-530 nm (optimally 518 nm) for EvaGreen; GreenFluorescent Dye (Lucigen); SYTO-13 dyes and 556 nm (optimally 560 nm) for SYTO-82 dye, the method of detection, change registration time starting from 14.93 minutes from the start of the reaction for Chlamydophila pneumoniae and the negative control.

### Example 5. Freeze-Drying Process

The method of preparation and freeze-drying of reagents for detecting the amplification and detection of the Chlamydophila pneumoniae MOMP gene using the oligonucleotides characterized in Example 1 with LAMP technology and the composition of the reaction mixture characterized in Example 2 with the temperature profile characterized in Example 4 and the detection method described in Example 5.

### Example 6. Description of the Freeze-Drying Process

The reaction components were mixed according to the composition described in Example 2, except the template DNA, to a total volume of 10 μl. The mixture was transferred to 0.2 ml tubes and subjected to the freeze-drying process according to the parameters below.

The mixture placed in the test tubes was pre-cooled to −80° C. for 2 hours. Then the freeze-drying process was carried out at the temperature of −80° C. for 3 hours under the pressure of 5-2 mBar.

### Example 7 Sensitivity of the Method

The sensitivity was determined by assaying serial dilutions of the standards: Genomic DNA from Chlamydophila pneumoniae Strain: CM-1 (VR-1360DQ™) with a minimum amount of 1 copy of bacteria per reaction mixture, where the product amplification was measured in real time-FIGS. 2, 4 (Real-Time LAMP for serial dilutions).

The time required to detect the emitted fluorescence for individual samples is shown in Tables 1, 2.

The characterized primers allow for the detection of Chlamydophila pneumoniae bacteria by detecting the MOMP gene fragment at a minimum number of 1 copy/reaction mixture.

TABLE 1
Time required to detect fluorescence for each dilution
of the Genomic DNA from Chlamydophila pneumoniae
Strain: CM-1 (VR-1360DQ ™) standard.

| | Time to exceed the baseline |
|---|---|
| Sample | fluorescence [min] |
| ChP NTC | Indefinite |
| ChP 1 copy | 18.54 |
| ChP 5 copies | 17.11 |
| ChP 10 copies | 17.04 |

TABLE 2
Time required to detect fluorescence for each dilution
of the Genomic DNA from Chlamydophila pneumoniae
Strain: CM-1 (VR-1360DQ ™) standard.

| | Time to exceed the baseline |
|---|---|
| Sample | fluorescence [min] |
| ChP NTC | Indefinite |
| ChP 10 copies | 17.07 |
| ChP 25 copies | 16.69 |
| ChP 50 copies | 16.34 |
| ChP 100 copies | 14.93 |

The superiority of the amplification method and the oligonucleotides described in this specification over the tests based on the RealTime-LAMP technology is due to the much higher sensitivity, which is shown in FIGS. 1, 3 and the reduction of the analysis time shown in FIGS. 2, 4 and Tables 1, 2.

1. A set of primers for amplifying the nucleotide sequence of the Chlamydophila pneumoniae MOMP gene, characterized in that it contains a set of internal primers with the following nucleotide sequences a) and b), as well as a set of external primers containing the following nucleotide sequences c) and d):
a) 5′ CGCTCCAAGAGAAAGAGGTGTC 3′ (SEQ ID NO: 3) or a sequence at least 90% identical to SEQ ID NO: 3-linked from the 3′ end, preferably by a TTTT bridge, with the sequence 5′ AAACGTTTCTTTAAGTAACGGAG 3′-(SEQ ID NO: 4) or a sequence at least 90% identical to SEQ ID NO: 4;
b) 5′ CTCGTGGAGCCTTATGGGAA 3′-(SEQ ID NO: 5) or a sequence at least 90% identical to SEQ ID NO: 5-linked from the 3′ end, preferably by a TTTT bridge, with the sequence 5′ GTGCATATTGGAATTCAGCT 3′-(SEQ ID NO: 6) or a sequence at least 90% identical to SEQ ID NO: 6;
c) 5′ CTGTAAATGCAAATGAACTACC 3′ (SEQ ID NO: 1) or a sequence at least 90% identical to SEQ ID NO: 1, and
d) sequence 5′ CACATTAAGTTCTTCAACTTTAGGT 3′ (SEQ ID NO: 2) or a sequence at least 90% identical to SEQ ID NO: 2.

2. The set of primers of claim 1, characterized in that it includes a loop primer sequence containing a nucleotide sequence contained in or complementary to the Chlamydophila pneumoniae MOMP gene (SEQ ID NO: 7)-5′ TGCGGTTGTGCAACTTTGGG 3′ or a sequence at least 90% identical to SEQ ID NO: 7.

3. A method of detecting Chlamydophila pneumoniae bacteria, characterized in that a selected region of the nucleic sequence of the bacterial genome is amplified using the set of primers as defined in claim 1, the amplification method being the LAMP method.

4. The method of detecting bacteria of claim 3, characterized in that the amplification is carried out with a temperature profile of:
−65.5° C., 40 min

5. The method of claim 4, characterized in that the end-point reaction is carried out with a temperature profile of 80° C., for additional 5 min.

6. A method for the detection of a Chlamydophila pneumoniae bacterium infection, characterized in that it comprises the detection method of claim 3.

7. A kit for the detection of Chlamydophila pneumoniae bacterium infection, characterized in that it comprises a set of primers as defined in claim 1.

8. The infection detection kit of claim 7, characterized in that it comprises 5.0 μl of WarmStart LAMP 2x Master Mix (NEB).

9. The infection detection kit of claim 7, wherein the primers have the following concentrations:
primer c) at 0.13 μM,
primer d) at 0.13 μM,
primer b) at 1.06 μM, and
primer a) at 1.06 μM.

10. A method of detecting Chlamydophila pneumoniae bacteria, characterized in that a selected region of the nucleic sequence of the bacterial genome is amplified using the set of primers as defined in claim 2, the amplification method being the LAMP method.

11. The method of detecting bacteria of claim 10, characterized in that the amplification is carried out with a temperature profile of:
−65.5° C., 40 min

12. The method of claim 11, characterized in that the end-point reaction is carried out with a temperature profile of 80° C., for additional 5 min.

13. A method for the detection of a Chlamydophila pneumoniae bacterium infection, characterized in that it comprises the detection method of claim 10.

14. A kit for the detection of Chlamydophila pneumoniae bacterium infection, characterized in that it comprises a set of primers as defined in claim 2.

15. The infection detection kit of claim 14, characterized in that it comprises 5.0 μl of WarmStart LAMP 2X Master Mix (NEB).

16. The infection detection kit of claim 14, wherein the primers have the following concentrations:
primer c) at 0.13 μM,
primer d) at 0.13 μM,
primer b) at 1.06 μM,
primer a) at 1.06 μM, and
the loop primer at 0.27 μM.

17. The infection detection kit of claim 9, comprising D-(+)-Trehalose dihydrate.

18. The infection detection kit of claim 9, comprising a fluorescent marker interacting with double-stranded DNA.

19. The infection detection kit of claim 14, comprising D-(+)-Trehalose dihydrate.

20. The infection detection kit of claim 14, comprising a fluorescent marker interacting with double-stranded DNA.


SEQUENCE LISTING (8 sequences)

SEQ ID NO: 1 - DNA, 22 bp, Artificial Sequence
  1 ChP MOMPF3
ctgtaaatgcaaatgaactacc

SEQ ID NO: 2 - DNA, 25 bp, Artificial Sequence
  2 ChP MOMPB3
cacattaagttcttcaactttaggt

SEQ ID NO: 3 - DNA, 22 bp, Artificial Sequence
  3 ChP MOMPF1c
cgctccaagagaaagaggtgtc

SEQ ID NO: 4 - DNA, 23 bp, Artificial Sequence
  4 ChP MOMPF2
aaacgtttctttaagtaacggag

SEQ ID NO: 5 - DNA, 20 bp, Artificial Sequence
  5  ChP MOMPB1c
ctcgtggagccttatgggaa

SEQ ID NO: 6 - DNA, 20 bp, Artificial Sequence
  6 ChP MOMPB2
gtgcatattggaattcagct

SEQ ID NO: 7 - DNA, 20 bp, Artificial Sequence
  7 ChP MOMPLoopB
tgcggttgtgcaactttggg

SEQ ID NO: 8 - DNA, 670 bp, Chlamydophila pneumoniae
gtagatagacctaacccggcctacaataagcatttacacgatgcagagtggttcactaatgccggcttcattgccttaaacatttgggatcgctttgatgttttctgtactttaggagcttctaatggttacattagaggaaactctacagcgttcaatctcgttggtttattcggagttaaaggtactactgtaaatgcaaatgaactaccaaacgtttctttaagtaacggagttgttgaactttacacagacacctctttctcttggagcgtaggcgctcgtggagccttatgggaatgcggttgtgcaactttgggagctgaattccaatatgcacagtccaaacctaaagttgaagaacttaatgtgatctgtaacgtatcgcaattctctgtaaacaaacccaagggctataaaggcgttgctttccccttgccaacagacgctggcgtagcaacagctactggaacaaagtctgcgaccatcaattatcatgaatggcaagtaggagcctctctatcttacagactaaactctttagtgccatacattggagtacaatggtctcgagcaacttttgatgctgataacatccgcattgctcagccaaaactacctacagctgttttaaacttaactgcatggaacccttctttactaggaaatg